(19) United States
(12) Patent Application Publication
(10) Pub. No.: US 2019/0336188 A1
(43) Pub. Date: Nov. 7, 2019

(54) Combined Protective Container and Delivery Device for Pedicle Screw

(71) Applicant: Spinal Balance, Inc., Toledo, OH (US)

(72) Inventors: Faezah Agarwal, Toledo, OH (US); Adam MacMillan, Toledo, OH (US); Aakash Agarwal, Toledo, OH (US); Daniel Mermuys, Toledo, OH (US); Marcel Ingels, Toledo, OH (US)

(73) Assignee: Spinal Balance, Inc., Toledo, OH (US)

(21) Appl. No.: 16/314,976

(22) PCT Filed: Jun. 29, 2017
(86) PCT No.: PCT/US2017/039886

§ 371 Date: Jan. 3, 2019

Related U.S. Application Data
(60) Provisional application No. 62/358,076, filed on Jul. 4, 2016.

(51) Int. Cl. A61B 17/86; A61B 17/70

(52) U.S. Cl. CPC A61B 17/865; A61B 2050/005; A61B 17/7082; A61B 17/7032

(57) ABSTRACT

A combined protective container and delivery device for an orthopedic implant device is provided. The combined protective container and delivery device includes an orthopedic implant device configured for insertion into a surgical site and a support configured to receive the orthopedic implant device. A base is configured to receive the orthopedic implant device and the support. A cover is configured to form a sealed container to prevent the ingress of contaminants to the orthopedic implant device and the support. The sealed container is configured for removal and insertion of the orthopedic implant device without manual handling of the orthopedic implant device

### BACKGROUND

A variety of orthopedic implant devices and procedures can be used in the repair of a skeletal system. The orthopedic implant devices may consist of screws, rods, plates, staples, spacers, pins, wires, and various combinations thereof. The orthopedic implant devices may be intended for short term or permanent implantation, and may be comprised of various materials, including the non-limiting examples of polymers, such as polyether ether ketone (PEEK), allograft bone, or implantable metals (such as titanium). As one example of an orthopedic implant device, spinal fixation devices are known for treating scoliosis, spondylolisthesis, degenerative disc disease, vertebra fractures, and other spinal disorders or abnormalities.

In many instances, spinal fixation devices include one or more pedicle screws, which can be individually secured to a vertebrae of a spine to provide anchor points that can then be connected together with a rod or other alignment or immobilization structure. A typical pedicle screw includes a threaded shaft portion having a yoke-shaped head portion extending therefrom. The threaded shaft portion is adapted to be secured to a vertebra of the spine, while the head portion is adapted to be connected to the rod or other alignment or immobilization structure.

As with all orthopedic implant devices, it is very important that the pedicle screw be sterilized prior to implantation within a patient. Traditional containers for pre-surgical packaging of pedicle screws may include a barrier system suitable for sterilization via irradiation, chemicals such as ethylene oxide, or steam. The barrier system can include gas permeable barriers, or non-permeable barriers, depending on sterilization method. In certain instances, the barrier systems will be provided with a plurality of layers to facilitate the transfer from a non-sterile environment to the sterile surgical environment.

However, single layer barrier systems may be used when direct transfer of the orthopedic implant device, including pedicle screws, from a non-sterile operator to a sterile operator is feasible. Commonly, the single layer barrier systems either contain a plurality of implant components, or can provide a single implant. Typically, the orthopedic device is taken from its container directly by a member of the surgical staff and handled directly following an aseptic technique. However, to maintain sterility, it is also very important that the amount of manual handling of the orthopedic implant device that occurs from the unsealing of the container to the implantation of the orthopedic implant device within the patient be minimized and completed in a controlled fashion.

It is known to keep orthopedic implant devices, including pedicle screws in sterile containers. Known sterile pedicle screw containers require an undesirably large amount of manual handling of the pedicle screw during the unsealing of the container to the implantation of the pedicle screw within the patient. Additionally, contact with the orthopedic implant device and the patient's own incision site can transfer bacteria within a patient. When inserting a bone screw into a vertebral body, for example, the surgeon typically applies the orthopedic implant device through a mid-line or posterior lateral incision to the spine. In this approach, the orthopedic device must pass through dermal, sub-dermal, and layers of musculature to reach the site of ultimate implantation. During this process, bacteria may be transferred into the cancellous bone of a vertebral body from the environment or from the patient's own dermal or sub dermal flora. This has potential to cause a deep tissue infection. This contact of the soft tissues with the orthopedic implant device can be even greater with larger patients. The ability to provide fixation over greater anatomical regions can often be even more difficult requiring multiple incisions, repeat handling of numerous orthopedic implant devices by the hospital staff, and greater opportunity for orthopedic implant devices to be exposed to infectious bacteria.

Thus, it would be advantageous to provide an improved protective container for pedicle screws and other orthopedic implant devices that minimizes the amount of manual handling thereof that occurs from the unsealing of the container to the implantation within the patient, and minimizes the contact of the orthopedic implant devices with areas other than the specific surgical site.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the combined protective container and delivery device for a pedicle screw.

The above objects as well as other objects not specifically enumerated are achieved by a combined protective container and delivery device for an orthopedic implant device. The combined protective container and delivery device includes an orthopedic implant device configured for insertion into a surgical site and a support configured to receive the orthopedic implant device. A base is configured to receive the orthopedic implant device and the support. A cover is configured to form a sealed container to prevent the ingress of contaminants to the orthopedic implant device and the support. The sealed container is configured for removal and insertion of the orthopedic implant device without manual handling of the orthopedic implant device.

Various objects and advantages of the combined protective container and delivery device for a pedicle screw will become apparent to those skilled in the art from the following detailed description, when read in light of the accompanying drawings.

### BRIEF DESCRIPTION OF THE DRAWINGS

FIG. 1 is an exploded perspective view of a first embodiment of a combined protective container and delivery device for a pedicle screw in accordance with this invention shown prior to assembly.

FIG. 2 is an exploded perspective view showing a pedicle screw prior to insertion within an interior cavity of a support of the combined protective container and delivery device for a pedicle screw of FIG. 1.

FIG. 3 is an exploded view, partially in side elevation and partially in perspective, showing the assembled support illustrated in FIG. 2 prior to insertion within an interior cavity of a base of the combined protective container and delivery device for a pedicle screw.

FIG. 4 is an exploded side elevational view showing the assembled base illustrated in FIG. 3 prior to the disposal of a cover thereon.

FIG. 5 is a side elevational view of a fully assembled container for a pedicle screw illustrated in FIGS. 1 through 4.

FIG. 6 is an exploded view, in side elevation of a second embodiment of a combined protective container and delivery device for a pedicle screw in accordance with this invention shown prior to assembly.

FIG. 7 is a side elevational view of a fully assembled container for a pedicle screw illustrated in FIG. 6.

FIG. 8 is an exploded view, in side elevation of a third embodiment of a combined protective container and delivery device for a pedicle screw in accordance with this invention shown prior to assembly.

FIG. 9 is a side view, of the combined protective container and delivery device for a pedicle screw illustrated in FIG. 8, shown with a support in an extended position.

FIG. 10A is a side view, of a fourth embodiment of a combined protective container and delivery device for a pedicle screw, shown with a support positioned within a base in an extended position.

FIG. 10B is a side view, of the combined protective container and delivery device for a pedicle screw of FIG. 10A, shown with a support removed from the base.

FIG. 11A is a side view of a first embodiment of a pedicle screw having a structured recess configured to receive a tool having a mating tip.

FIG. 11B is a side view of a second embodiment of a pedicle screw having a structured recess configured to receive a tool having a mating tip.

FIG. 11C is a side view of a second embodiment of a pedicle screw having a structured recess configured to receive a tool having a mating tip.

FIG. 12A is a side view of an embodiment of a support having a plurality of apertures and a plurality of spaced apart recesses.

FIG. 12B is a side view of the support of FIG. 12A illustrating a pedicle screw contained therein.

FIG. 13A is a side view of an embodiment of a support having a window and a tapered tip.

FIG. 13B is a side view of the support of FIG. 13A illustrating a pedicle screw in an initial position.

FIG. 13C is a side view of the support of FIG. 13A illustrating a pedicle screw in a second position.

FIG. 13D is a side view of the support of FIG. 13A illustrating a pedicle screw in a final position.

FIG. 14A is a side view, in cross-section of an embodiment of a support having an annular protrusion.

FIG. 14B is a side view, in cross-section of an embodiment of a pedicle screw having an annular groove.

FIG. 14C is a side view, in cross-section, of the support of FIG. 14A assembled with the pedicle screw of FIG. 14B.

FIG. 15A is a side view of a support having opposing portions assembled together.

FIG. 15B is a side view of the support of FIG. 15A showing the opposing portions broken apart.

FIG. 16A is a side view of a support having opposing portions assembled together with a framework.

FIG. 16B is a side view of the support of FIG. 16A showing rotation of one of the opposing portions relative to the other portion.

FIG. 17 is a side view of a support configured to provide unencumbered movement of a pedicle screw therethrough.

FIG. 18 is a side view of a support having features in a tapered tip.

FIG. 19A is a side view of another embodiment of a container having a base, a carrier and a pedicle screw.

FIG. 19B is a perspective view of a portion of the container of FIG. 19A illustrating rotation of the base and longitudinal movement of the carrier.

FIG. 19C is a side view, in elevation, of the container of FIG. 19A illustrating retention of the carrier in the base after the pedicle screw has been removed.

FIG. 20A is a side view, in elevation, of another embodiment of a support used in cooperation with a tool.

FIG. 20B is a side view, in elevation, of another embodiment of a support used in cooperation with a tool.

### DETAILED DESCRIPTION

The combined protective container and delivery device for a pedicle screw will now be described with occasional reference to the specific embodiments. The combined protective container and delivery device for a pedicle screw may, however, be embodied in different forms and should not be construed as limited to the embodiments set forth herein. Rather, these embodiments are provided so that this disclosure will be thorough and complete, and will fully convey the scope of the combined protective container and delivery device for a pedicle screw to those skilled in the art.

Unless otherwise defined, all technical and scientific terms used herein have the same meaning as commonly understood by one of ordinary skill in the art to which the combined protective container and delivery device for a pedicle screw belongs. The terminology used in the description of the combined protective container and delivery device for a pedicle screw herein is for describing particular embodiments only and is not intended to be limiting of the combined protective container and delivery device for a pedicle screw. As used in the description of the combined protective container and delivery device for a pedicle screw and the appended claims, the singular forms “a,” “an,” and “the” are intended to include the plural forms as well, unless the context clearly indicates otherwise.

Unless otherwise indicated, all numbers expressing quantities of dimensions such as length, width, height, and so forth as used in the specification and claims are to be understood as being modified in all instances by the term “about.” Accordingly, unless otherwise indicated, the numerical properties set forth in the specification and claims are approximations that may vary depending on the desired properties sought to be obtained in embodiments of the present invention. Notwithstanding that the numerical ranges and parameters setting forth the broad scope of the combined protective container and delivery device for a pedicle screw are approximations, the numerical values set forth in the specific examples are reported as precisely as possible. Any numerical values, however, inherently contain certain errors necessarily resulting from error found in their respective measurements.

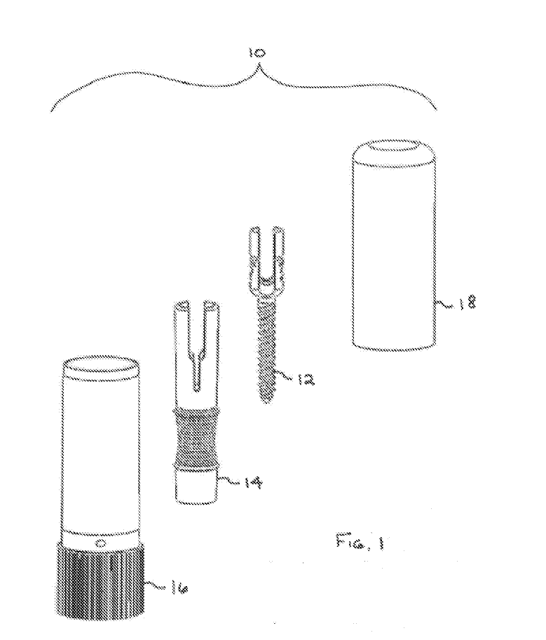

Referring now to FIG. 1, an exploded perspective view of a container is illustrated generally at 10. Generally, the container 10 is configured to provide a sterilized orthopedic implant device that minimizes the amount of manual handling of the orthopedic implant device that occurs from the unsealing of the container 10 to the implantation within the patient. The container 10 provides a packaging and implant delivery housing that can maintain the sterility of the orthopedic implant device prior to use, while preventing contamination of the orthopedic implant device until the ultimate delivery to the surgical site. The container 10 is designed such that the surgeon or other user of the orthopedic implant device can grab the container 10 with a contaminated glove or hand, while allowing access to the orthopedic implant device using either a tool or a clean hand. This improves upon traditional sterile orthopedic implant device packaging that requires the user to carefully grab an inner package or the implant directly from an outer sterile barrier system. The container 10 includes a pedicle screw 12, a support 14, a base 16 and a cover 18.

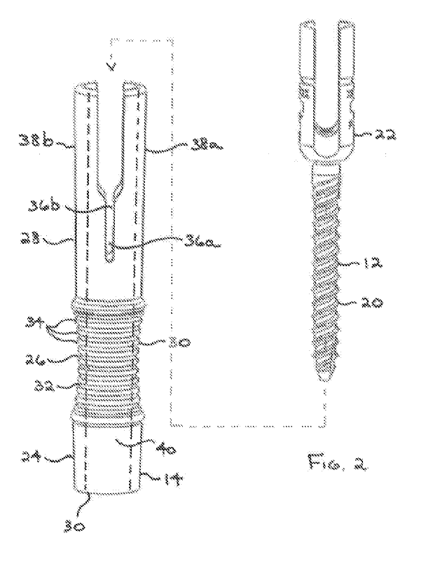

Referring now to FIG. 2, the pedicle screw 12 is conventional in the art and is intended to be representative of any orthopedic implant device or other medical implant or device that may be sterilized and implanted within a patient. Thus, it will be appreciated that the pedicle screw 10 is merely illustrative of one application for this invention and, therefore, forms no part of the invention itself. The pedicle screw 12 includes a threaded shaft portion 20 and has a yoke-shaped head portion 22 extending therefrom. The threaded shaft portion 20 is adapted to be secured to a vertebra of the spine, while the head portion 22 is adapted to be connected to the rod or other alignment or immobilization structure (not shown).

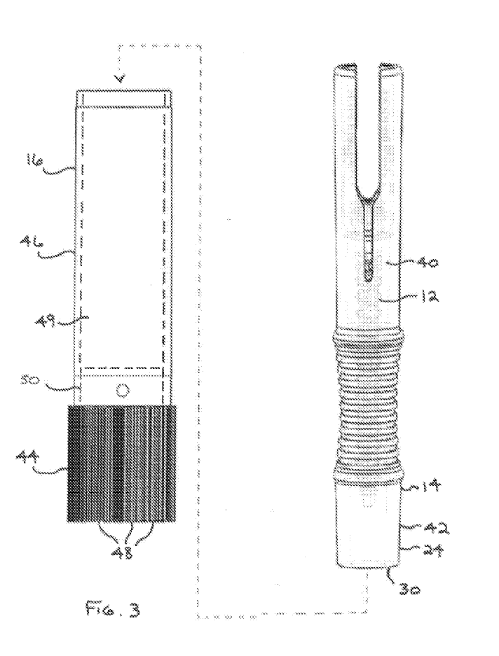

Referring again to FIG. 2, the support 14 forms a generally hollow, cylindrically shaped structure and includes a lower portion 24, an intermediate portion 26, and an upper portion 28. The lower portion 24 includes a bottom end 30. In certain embodiments, the bottom end 30 includes an aperture (not shown) such that the bottom end 30 is open (as shown in FIG. 3). The aperture is configured to allow surgical devices to extend through the support 14. However, it should be appreciated that the aperture is optional and not required for operation of the container 10.

Referring again to the embodiment illustrated in FIG. 2, the intermediate portion 26 has an outer surface 30 forming an hourglass-shaped contour. The outer surface 30 has a plurality of external annular ridges 34 provided thereon. The hourglass-shaped contour of the outer surface 30 and the external annular ridges 34 cooperate to provide a positive gripping surface. However, it should be appreciated that in other embodiments, the outer surface 30 can have other contours and other surface features sufficient to provide a positive gripping surface. It should further be appreciated that the hourglass-shaped contour of the outer surface 30 and the external annular ridges 34 are optional and not required for operation of the container 10.

Referring again to the embodiment illustrated in FIG. 2, the upper portion 28 includes a plurality of aligned slots 36a, 36b configured to form a pair of opposed arms 38a, 38b. The opposed arms 38a, 38b will be discussed in more detail below. While the embodiment shown in FIG. 2 illustrates the slots 36a, 36b and the arms 38a, 38b, in other embodiments, the upper portion 28 can be formed from other structures. Accordingly, it should be appreciated that the aligned slots 36a, 36b and the opposed arms 38a, 38b are not required for operation of the container 10.

Referring again to FIG. 2, the hollow structures of the lower portion 24, intermediate portion 26 and upper portion 28 cooperate to define an interior cavity 40 extending therethrough. The pedicle screw 12 is inserted into the interior cavity 40 of the support 14. In certain embodiments, the pedicle screw 12 may be frictionally engaged by the pair of opposed arms 38a, 38b of the upper portion 28 of the support 14. Alternatively, the pedicle screw 12 can be engaged by other portions of the support 14. It is also contemplated that the interior cavity 40 extending through the support 14 can include a device-specific geometry or other engaging structures configured to securely engage a pedicle screw or other orthopedic implant device. Non-limiting examples of engaging structures can include interference fits, threads, tongue and groove, snap fits, breakaway tabs or the like. Non-limiting examples of other orthopedic implant devices include interbody cages, spinal spacers, arthroplasty disks and the like. Insertion of the pedicle screw 12 into the interior cavity 40 of the support 14 forms an assembled support 42 as shown in FIG. 3.

Referring now to FIG. 3, the base 16 is generally hollow and cylindrical in shape. The base 16 includes a lower portion 44 and an upper portion 46. The lower portion 44 includes a plurality of longitudinal ridges 48 arranged in a circumferential orientation. The longitudinal ridges 48 are configured to provide a positive grip surface as the lower portion 44 is rotated relative to the upper portion 46. While the embodiment illustrated in FIG. 3 illustrates the use of the longitudinal ridges 48, in alternate embodiments, other surface structures can be used sufficient to provide a positive grip surface as the lower portion 44 is rotated relative to the upper portion 46.

Referring again to FIG. 3, the hollow structures of the lower portion 44 and the upper portion 46 of the base 16 cooperate to define an interior cavity 49 extending therethrough. An internal platform 50 is positioned within the interior cavity 49 and is configured for longitudinal movement within the interior cavity 49 relative to the lower portion 44. Longitudinal movement of the internal platform 50 is actuated by rotation of the lower portion 44 relative to the upper portion 46. The longitudinal movement of the internal platform 50 will be discussed in more detail below.

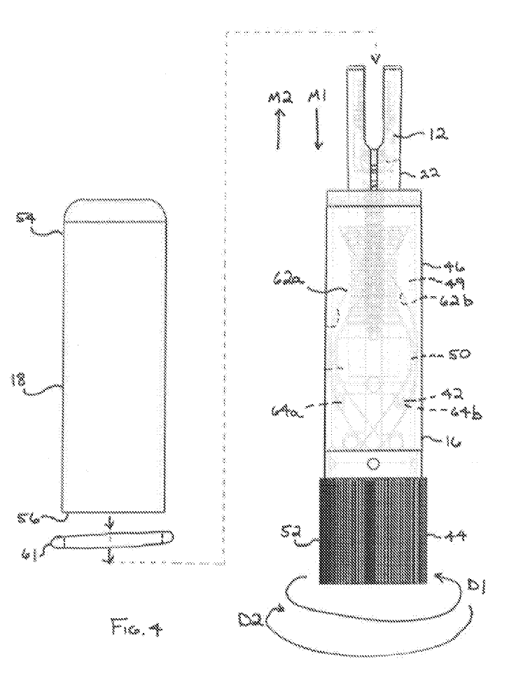

Referring again to FIG. 3, the assembled support 42 is inserted into the interior cavity 49 of the base 16 until the bottom end 30 of the lower portion 24 of the assembled support 42 engages the internal platform 50. Insertion of the assembled support 42 into the interior cavity 49 of the base 16 forms an assembled base 52 as shown in FIG. 4.

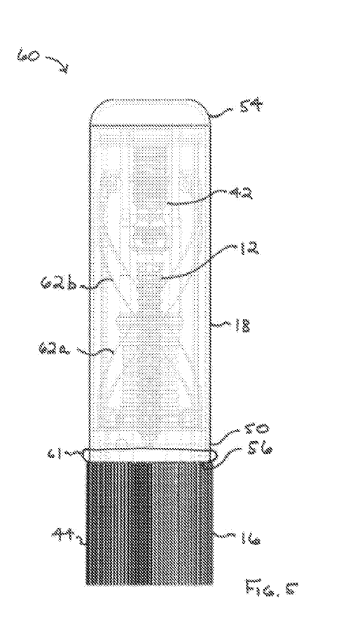

Referring now to FIGS. 4 and 5, the cover 18 has a closed upper end 54 and an open lower end 56. The cover 18 may be disposed about the upper portion 46 of the base 16. When the cover 18 is disposed about the upper portion 46 of the base 16, the lower opened end 56 thereof is disposed about and engages (frictionally or otherwise as desired) an outer surface of a portion of the base 16. Disposing the cover about the upper portion 46 of the base 16 forms an assembled container 60 as shown in FIG. 5.

Referring again to FIGS. 4 and 5, optionally a seal 61 can be positioned between the base 16 and the cover 18. The seal 61 is configured to seal the assembled container 60 such as to prevent the ingress of contaminants. In the illustrated embodiment, the seal 61 has the form of an O-ring and can be formed from any desired material sufficient to prevent the ingress of contaminants. In other embodiments, the base 16 and the cover 18 can be sealed by other structures, mechanisms and devices, including the non-limiting examples of mating threads, inference fits, gaskets, break-away tabs and the like. In certain instance, the seal 61 can have the form of a tamper-evident seal. The tamper-evident seal can be configured to prevent the removal, handling, and replacement of the pedicle screw 12 and/or support 14 back into the container 60.

Referring again to FIGS. 4 and 5, the internal platform 50 is movable within the interior cavity 49 between an extended position (as shown in FIG. 4) and a contracted position (as shown in FIG. 5). The extended position is configured such that the assembled support 42, containing the pedicle screw 12, is accessible. The contracted position is configured such that the assembled support 42, containing the pedicle screw 12 is sealed within the assembled container 60, thereby preventing the ingress of contaminants.

Referring again to FIGS. 4 and 5, movement of the assembled support 42 is actuated by rotation of the lower portion 44 of the base 16. When the lower portion 44 of the base 16 is rotated in a first direction, as indicated by direction arrow D1, relative to the upper portion 46 of the base 16, the internal platform 50 and the engaged assembled support 42 are moved longitudinally toward the contracted position as shown in FIG. 5, as indicated by direction arrow M1. Conversely, when the lower portion 44 of the base 16 is rotated in a second direction (opposite to the first direction), as indicated by direction arrow D2, relative to the upper portion 46 of the base 16, the internal platform 50 is moved longitudinally toward the extended position as shown in FIG. 4, as indicated by direction arrow M2.

Referring again to FIGS. 4 and 5, the longitudinal movement of the internal platform 50 and the engaged assembled support 42 are accomplished with the use of a plurality of helical grooves 62a, 62b provided on an inner surface of the upper portion 46 of the base 16. The internal platform 50 is equipped with a plurality of tabs 64a, 64b configured to extend into the helical grooves 62a, 62b. The internal platform 50 is connected to the lower portion 44 of the base 16 for rotation therewith. As a result, when the lower portion 44 of the base 16 is rotated relative to the upper portion 46 as described above, the tabs 64a, 64b cooperate with the helical grooves 62a, 62b to effect longitudinal movement of the internal platform 50 between the contracted and extended positions, as described above. While the embodiment shown in FIGS. 4 and 5 illustrate the use of a quantity of two helical grooves 62a, 62b and a quantity of two tabs 64a, 64b, in other embodiments, any desired quantity of grooves and associated tabs can be used sufficient to effect longitudinal movement of the internal platform 50 between the contracted and extended positions. In still other embodiments, it is within the contemplation of the invention that other structure or combination of structures may be provided to effect longitudinal movement of the internal platform 50 between the contracted and extended positions.

Referring again to FIGS. 4 and 5, the process for removing the pedicle screw 12 from the assembled container 60 device will now be described. In an initial step, the cover 18 is removed from the upper portion 46 of the base 16. In a subsequent step, the lower portion 44 of the base 16 is rotated in a second direction, as indicated by direction arrow D2, relative to the upper portion 46, thereby causing the internal platform 50 to be moved longitudinally within the interior cavity 49, as indicated by direction arrow M2, to the extended position near the upper portion 46, as shown in FIG. 4. Such movement of the internal platform 50 and the engaged assembled support 42 exposes an upper portion of the pedicle screw 12, which can then be engaged by an installation tool (not shown) while the lower portion of the pedicle screw 12 continues to remain supported by the support 14 within the base 16. Advantageously, the processes for unsealing the assembled container 60, removing the pedicle screw 12 from the assembled container 60 and implantation of the pedicle screw 12 requires no manual handling of the pedicle screw 12.

Referring again to FIG. 1, the support 14, base 16 and the cover 18 can be formed from flexible, semi-rigid or rigid materials or combinations of flexible, semi-rigid or rigid materials, including the non-limiting examples of polymeric materials, plastic-based materials or metallic materials. The support 14, base 16 and the cover 18 can be formed using manufacturing processes such as molding, machining, printing using 3D printers and the like. It is also contemplated that the support 14, base 16 and the cover 18 may be formed as unitary bodies or as a plurality of discrete components that are subsequently formed into the intended structures. The plurality of discrete components may be assembled by spin welding, ultrasonic welding, chemical bonding, mechanical interaction or the like.

While the embodiment of the container 10 described above is illustrated with a pedicle screw, it is contemplated that other orthopedic implant devices can be housed within the container 10. It is also contemplated that more than one orthopedic implant devices can be housed within the container 10 and that the various orthopedic implant devices can be the same device or devices having different sizes, shapes and configurations.

While the embodiment of the container 10 described above involves the movement of the internal platform 50 from a contracted position to an extended position and from an extended position to a contracted position, in other embodiments it is contemplated that the container 10 may be configured such that the pedicle screw 12 can only be advanced in an outward direction from the base 16, and not subsequently retracted back therein. That is, it is contemplated that the actuation of the base 16 only serves to extend the pedicle screw 12 in one direction. This feature may be provided to prevent potential contamination of the pedicle screw 12 due to re-use of the container 10.

While the embodiment of the assembled container 60 shown in FIG. 5 has been described above as being a sealed structure, it is contemplated that in other embodiments, the assembled container 60 may further be encased in one or more additional sterile barriers. Non-limiting examples of additional sterile barriers can include tubes, pouches, trays, vials or the like. The use of the additional sterile barriers is contemplated to allow for additional transfers of the assembled container 60 from non-sterile environments to other sterile environments.

While the embodiment of the assembled container 60 is shown in FIG. 5 as having a generally circular cross-sectional shape, it is contemplated that the assembled container 60 can have a non-circular cross-sectional shape configured to discourage rolling of the assembled container 60. It is also contemplated that the assembled container 60 can include an exterior feature configured to discourage rolling. As one non-limiting example, the assembled container 60 can include one or more projections on an exterior surface.

In the embodiment illustrated in FIG. 5, the pedicle screw 12 and support 14 are packaged within the container 60. However, it should be appreciated that in other embodiments, additional apparatus can also be packaged within the container 60. As one non-limiting example, one or more tools (not shown) can be packaged within the container 60, with the one or more tools adapted to facilitate removal and/or implantation of the pedicle screw 12. Advantageously, packaging one or more tools in the container 60 maintains the entirety of the pedicle screw 12 and the support 14 from contacting any patient related or surgically related surfaces other than the specific surgical site for the pedicle screw 12. In still other embodiments, the container 60 can include a depth gage and relative depth markings for the pedicle screw 12 and/or k-wire components to provide feedback to the surgeon on the depth of the pedicle screw 12 within the patient or surgical site.

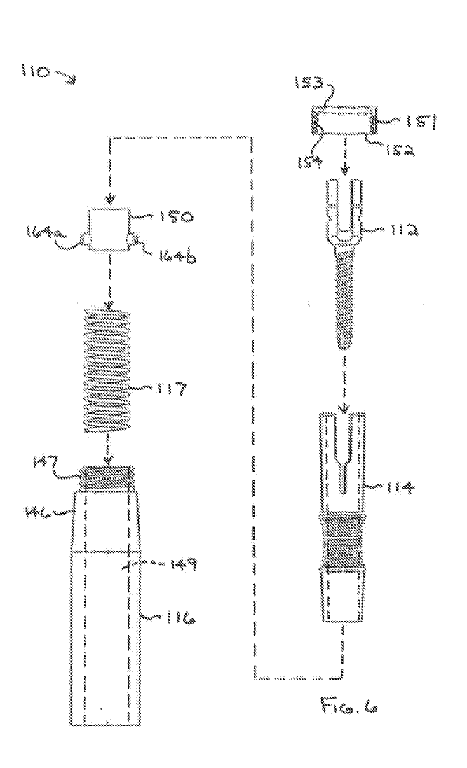

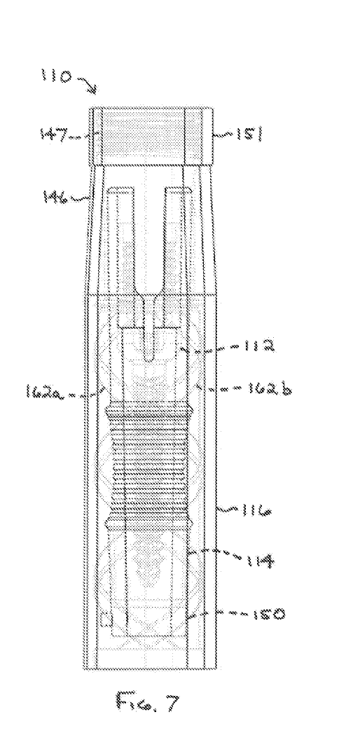

Referring now to FIGS. 6 and 7, a second embodiment of a container is illustrated generally at 110. Generally, the container 110 is a spring-loaded device configured to provide an orthopedic implant device that minimizes the amount of manual handling of the orthopedic implant device that occurs from the unsealing of the container 110 to the implantation within the patient. The container 110 includes a pedicle screw 112, a support 114, a base 116, a spring element 117, an internal platform 150 and a cap 151.

Referring again to the embodiment shown in FIGS. 6 and 7, the pedicle screw 112 and the support 114 are the same as, or similar to the pedicle screw 12 and the support 14 illustrated in FIG. 2 and described above. In other embodiments, the pedicle screw 112 and the support 114 can be different from the pedicle screw 12 and the support 14.

Referring again to FIGS. 6 and 7, the base 116 includes an interior cavity 149 and optionally a plurality of helical grooves 162a, 162b, as shown in FIG. 7. In the embodiment shown in FIG. 7, the optional helical grooves 162a, 162b are the same as, or similar to the helical grooves 62a, 62b illustrated in FIG. 4 and described above. In other embodiments, helical grooves 162a, 162b can be different from the helical grooves 62a, 62b. The base 116 further includes an upper portion 146 and a threaded extension 147 extending from the upper portion 146.

Referring again to FIGS. 6 and 7, the spring element 117 is configured for several functions. First, the spring element is configured for positioning within the interior cavity 149 of the base 116. Second, the spring element 117 is configured for contact with the internal platform 150. Third, the spring element 117 is configured for a compressed first orientation and finally, the spring element 117 is configured for an extended second orientation. In the illustrated embodiment, the spring element 117 has the form of a compression spring. Alternatively, the spring element 117 can have other forms sufficient for the functions discussed above.

Referring again to FIGS. 6 and 7, the internal platform 150 is positioned within the internal cavity 149 of the base 116 and seats against an upper surface of the spring element 117. The internal platform 150 includes a plurality of optional tabs 164a, 164b. In the illustrated embodiment, the tabs optional 164a, 164b are the same as, or similar to the tabs 64a, 64b illustrated in FIG. 4 and described above. In other embodiments, tabs 164a, 164b can be different from the tabs 64a, 64b.

Referring now to FIG. 6, the cap 151 includes an open end 152 and a closed end 153. The open end 152 includes an internally threaded segment 154 configured for threaded attachment to the threaded extension 147 extending from the upper portion 146 of the base 116.

Referring again to FIG. 6, the container 110 is assembled as follows. In an initial step, the spring element 117 is positioned within the internal cavity 149 of the base 116 such that the spring element 117 longitudinally aligns with the internal cavity 149. In a next step, the internal platform 150 is positioned within the internal cavity 149 of the base 116 and is seated against an upper surface of the spring element 117. The internal platform 150 is positioned such that the optional tabs 164a, 164b are received by the optional helical grooves 162a, 162b in the base 116. Next, the pedicle screw 112 is seated in the support 114 in the same manner as described above. The assembled pedicle screw 112 and support 114 are inserted into the internal cavity 149 of the base 116 such that the support 114 seats against the internal platform 150. In a next step, the assembled pedicle screw 112 and support 114 are further urged into the internal cavity 149 of the base 116 a distance until the spring element 117 is compressed and the cap 151 can be threaded onto the threaded extension 147 extending from the upper portion 146 of the base 116, as shown in FIG. 7. In a final step, the cap 151 is tightened on the threaded extension 147 extending from the upper portion 146 of the base 116, thereby forming a sealed container 110.

Referring now to FIG. 7, the container 110 remains sealed with the cap 151 tightened on the threaded extension 147 extending from the upper portion 146 of the base 116 until such time that the cap 151 is removed. At that time, the spring mechanism 117 urges the internal platform 150 in a direction toward the threaded extension 147. In turn, movement of the internal platform 150 urges the engaged assembled pedicle screw 112 and support 114 into an exposed position, which can then be engaged by an installation tool (not shown) while the lower portion of the pedicle screw 112 continues to remain supported by the support 114 within the base 116. Advantageously, the processes for unsealing the spring-loaded container 110, removing the pedicle screw 112 from the container 110 and implantation of the pedicle screw 112 requires no manual handling of the pedicle screw 112.

While the embodiment of the container 110 shown in FIGS. 6 and 7 is described above as having optional helical grooves 162a, 162b and optional tabs 164a, 164b, it should be appreciated that in other embodiments, the container 110 can omit the grooves 162a, 162b and tabs 164a, 164b and function as described above. In certain instances, the optional helical grooves 162a, 162b and the tabs 164a, 146b can be configured to reduce the speed, or increase the time, for moving the assembled pedicle screw 112 and support 114 into an exposed position, thereby provided controlled movement of the assembled pedicle screw 112 and support 114.

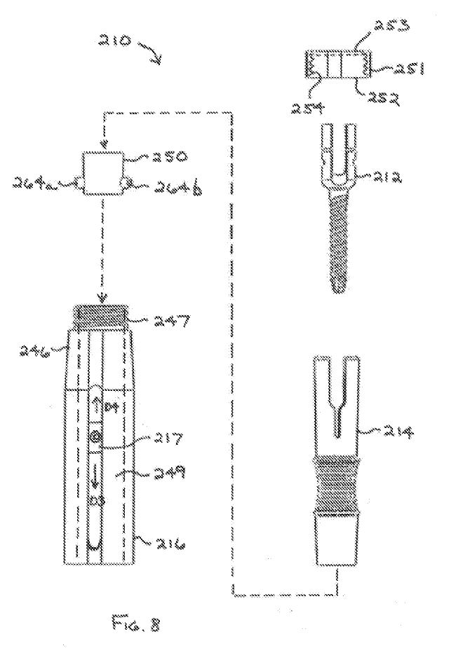

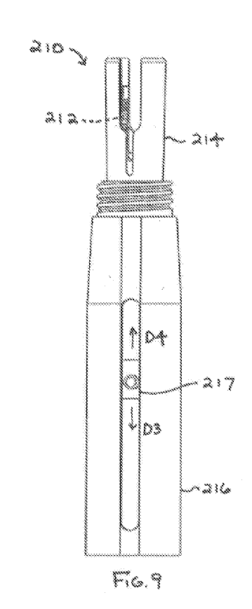

Referring now to FIGS. 8 and 9, a third embodiment of a container is illustrated generally at 210. Generally, the container 210 includes a linear translation device for actuating movement of an orthopedic implant device from a contracted position to an extended position and from the extended position to the contracted position, while minimizing the amount of manual handling of the orthopedic implant device that occurs from the unsealing of the container 210 to the implantation within the patient. The container 210 includes a pedicle screw 212, a support 214, a base 216, an internal platform 250 and a cap 251.

Referring again to the embodiment shown in FIGS. 8 and 9, the pedicle screw 212 and the support 214 are the same as, or similar to the pedicle screw 12 and the support 14 illustrated in FIG. 2 and described above. In other embodiments, the pedicle screw 212 and the support 214 can be different from the pedicle screw 12 and the support 14.

Referring again to FIGS. 8 and 9, the base 216 includes an interior cavity 249, an optional plurality of helical grooves (not shown for purposes of clarity), an upper portion 246 and a threaded extension 247 extending from the upper portion 246. In the illustrated embodiment, the interior cavity 249, helical grooves, upper portion 246 and threaded extension 247 are the same as, or similar to the an interior cavity 149, plurality of helical grooves 162a, 162b, upper portion 146 and threaded extension 147 extending from the upper portion 146 illustrated in FIGS. 6-7 and described above. However, in other embodiments, the interior cavity 249, helical grooves, upper portion 246 and threaded extension 247 can be different from the interior cavity 149, plurality of helical grooves 162a, 162b, upper portion 146 and threaded extension 147 extending from the upper portion 146. The base 216 also includes a linear translation device 217. The linear translation device 217 is configured for sliding movement along a portion of the base 216. The linear translation device 217 will be discussed in more detail below.

Referring again to FIGS. 8 and 9, the internal platform 250 is positioned with the internal cavity 249 of the base 216 and seats against a portion of the linear translation device 217. The internal platform 250 includes a plurality of optional tabs 264a, 264b. In the illustrated embodiment, the optional tabs 264a, 264b are the same as, or similar to the tabs 64a, 64b illustrated in FIG. 4 and described above. In other embodiments, tabs 264a, 264b can be different from the tabs 64a, 64b.

Referring again to FIGS. 8 and 9, the cap 251 includes an open end 252 and a closed end 253. The open end 252 includes an internally threaded segment 254 configured for threaded attachment to the threaded extension 247 extending from the upper portion 246 of the base 216.

Referring again to the embodiment shown in FIGS. 8 and 9, the linear translation device 217 is configured for several functions. First, the linear translation device 217 is configured such that a portion of the linear translation device extends into the interior cavity 249 of the base 216 and another portion of the linear translation device 217 extends through the base 216. Second, the linear translation device 217 is configured for contact with the internal platform 250. Third, the linear translation device 217 is configured for slidable movement, as shown be reference arrows D3, D4, from a first orientation to a second orientation. Finally, the linear translation device 217 is configured such that movement from the first orientation to the second orientation results in corresponding movement of the internal platform 250. In the illustrated embodiment, the linear translation device 217 has the form of a sliding switch. Alternatively, the linear translation device 217 can have other forms sufficient for the functions discussed above.

Referring now to FIG. 8, the container 210 is assembled as follows. In an initial step, the linear translation device 217 is positioned within the internal cavity 249 of the base 216. In a next step, the internal platform 250 is positioned within the internal cavity 249 of the base 216 and is seated against, and connected to, a portion of the linear translation device 217. The internal platform 250 is positioned such that the optional tabs 264a, 264b are received by the optional helical grooves in the base 216. Next, the pedicle screw 212 is seated in the support 214 in the same manner as described above. The assembled pedicle screw 212 and support 214 are inserted into the internal cavity 249 of the base 216 such that the support 214 seats against the internal platform 250. In a next step, the linear translation device 217 is slidably moved to the contracted position, thereby allowing the cap 251 to be threaded onto the threaded extension 247 extending from the upper portion 246 of the base 216, as shown in FIG. 9. In a final step, the cap 251 is tightened on the threaded extension 247 extending from the upper portion 246 of the base 216, thereby forming a sealed container 210.

Referring now to FIG. 9, the container 210 is illustrated with the cap removed and the assembled pedicle screw 212 and support 214 in the extended position as a result of slidable movement of the linear translation device 217. The slidable movement of the linear translation device 217 has urged the internal platform 250 in a direction toward the threaded extension 247. In turn, movement of the internal platform 250 has urged the engaged assembled pedicle screw 212 and support 214 into the exposed position, which can then be engaged by an installation tool (not shown) while the lower portion of the pedicle screw 212 continues to remain supported by the support 214 within the base 216. Advantageously, the processes for unsealing the container 210, removing the pedicle screw 212 from the container 210 and implantation of the pedicle screw 212 requires no manual handling of the pedicle screw 212.

While the embodiment of the container 210 shown in FIGS. 8 and 9 is described above as having optional helical grooves and optional tabs 264a, 264b, it should be appreciated that in other embodiments, the container 210 can omit the grooves and tabs 264a, 264b and function as described above. In certain instances, the optional helical grooves and the tabs 264a, 246b can be configured to reduce the speed, or increase the time, for moving the assembled pedicle screw 212 and support 214 into an exposed position, thereby provided controlled movement of the assembled pedicle screw 212 and support 214.

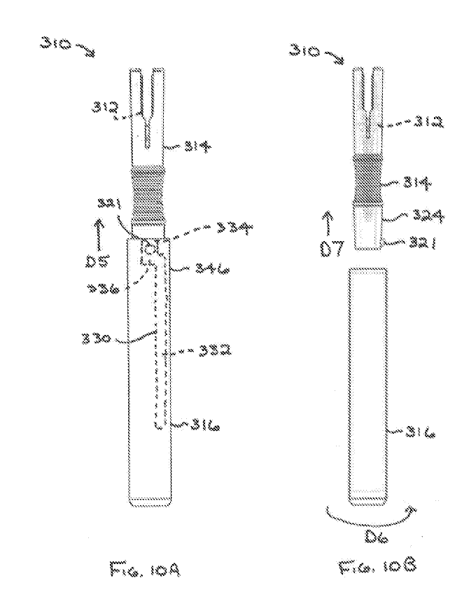

Referring now to FIGS. 10A and 10B, a fourth embodiment of a container is illustrated generally at 310. Generally, the container 310 includes one or more structures or devices configured to prevent an assembled pedicle screw and support from accidently falling out of the container 310 in the event the container 310 is opened with an upper portion of a base pointed in a downward direction. The container 310 includes a pedicle screw 312, a support 314, and a base 316.

Referring again to the embodiment shown in FIGS. 10A and 10B, the pedicle screw 312 is the same as, or similar to the pedicle screw 12 illustrated in FIG. 2 and described above. In other embodiments, the pedicle screw 312 can be different from the pedicle screw 12.

Referring again to the embodiment shown in FIGS. 10A and 10B, the support 314 is the same as, or similar to the support 314 illustrated in FIG. 2 and described above with the exception that the support 314 includes a detent 321 extending from an outer surface of a lower portion 324. The detent 321 will be discussed in more detail below.

Referring again to FIGS. 10A and 10B, the base 116 is the same as, or similar to the base 16 illustrated in FIG. 2 and described above with the exception that the base 316 includes an internal path 330 configured to receive and guide the detent 321 extending from an outer surface of the lower portion 324. The internal path 330 includes first and second vertical elements 332, 334 and at least one horizontal element 336. The first vertical element 332 is configured to guide the detent 321 as the assembled pedicle screw 312 and support 314 is moved toward the upper portion 346 of the base 316, as represented by direction arrow D5. Once the detent 321 encounters the horizontal element 336 of the internal path 330, the assembled pedicle screw 312 and support 314 cannot continue to move in the outward direction D5. As shown in FIG. 10b, the base 316 must be rotated in direction D6, thereby allowing the detent 321 to be guided in the horizontal element 336 until the second vertical element 336 is encountered. Once the rotating detent 321 encounters the second vertical element 336, the assembled pedicle screw 312 and support 314 is free to move in the outward direction D7 such as to be completely removed from the base 316. Advantageously, the structures of the detent 321 extending from an outer surface of the lower portion 324 and the internal path 330 in the base 312 cooperate to prevent the assembled pedicle screw 312 and support 314 from accidently falling out of the container 310 in the event the container 310 is opened with the upper portion 346 of the base 316 pointed in a downward direction.

While the embodiment illustrated in FIGS. 10A and 10B shows the cooperating structures of the detent 321 and the internal path 330, it should be appreciated that other structures, mechanisms and devices can be used, sufficient to prevent the assembled pedicle screw 312 and support 314 from accidently falling out of the container 310 in the event the container 310 is opened with the upper portion 346 of the base 316 pointed in a downward direction. Non-limiting examples of cooperating structures include keys, projections, knobs, partial threads, interference fits, snap rings, racks, tongue and groove features and the like.

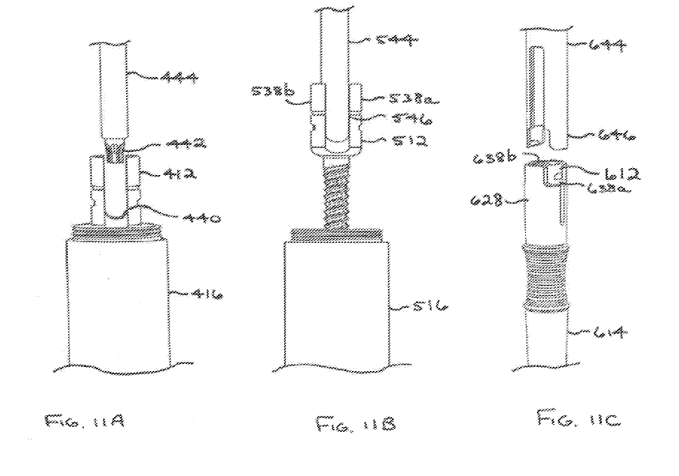

Referring now to FIGS. 11A-11C for a fifth embodiment of a container, it is within the contemplation of the container that a pedicle screw can be removed from a support and from a base with an apparatus or tool, thereby eliminating the need for human contact with the pedicle screw and maintaining the sterility of the pedicle screw. In such instances, the structure of the pedicle screw and the apparatus or tool can be configured to cooperate to facilitate removal of the pedicle screw by the apparatus or tool. Referring now to FIG. 11A in a first non-limiting example, a pedicle screw 412 and base 416 are illustrated (the support is omitted for purposes of clarity). In the illustrated embodiment, the base 416 is the same as, or similar to, the base 16 illustrated in FIG. 3 and described above. However, in other embodiments, the base 416 can be different than the base 16.

Referring again to FIG. 11A, the pedicle screw 412 is the same as, or similar to, the pedicle screw 12 illustrated in FIG. 2 and described above with the exception that the pedicle screw 412 includes a structured recess 440 configured to receive a tool tip 442 of a tool 444 having a mating structure. As one non-limiting example, the tool 444 has a tool tip with the form of an Allen-head or torx-head socket and the structured recess 440 of the pedicle screw 412 has a mating structure. In operation, the tool tip 442 is inserted into the structured recess 440 of the pedicle screw 412 and the tool 444 is used to remove the pedicle screw 412 from the container. Advantageously, the tool 444 facilitates removal of the pedicle screw 412 from the support and from the base without the need for human contact, thereby maintaining the sterility of the pedicle screw.

Referring now to FIG. 11B in another non-limiting example, the pedicle screw 512 is the same as, or similar to, the pedicle screw 12 illustrated in FIG. 2 and described above. The pedicle screw 512 includes opposing arms 538a, 538b, with each of the arms 538a, 538b having an interior surface. The interior surfaces of the opposing arms 538a, 538b forms a gap therebetween, with the gap having a circular cross-sectional shape. A tool 544 is provided that includes a tool tip 546. The tool tip 546 has a circular cross-sectional shape with a diameter such as to cause a frictional fit with the interior surfaces of the opposing arms 538a, 538b. In operation, the tool tip 544 is inserted between the interior surfaces of the opposing arms 538a, 538b and the friction fit facilitates removal of the pedicle screw 512 by the tool 544. Advantageously, the tool 544 facilitates removal of the pedicle screw 512 from the support and from the base without the need for human contact, thereby maintaining the sterility of the pedicle screw.

Referring now to FIG. 11C in another non-limiting example, the pedicle screw 612 is shown within the support 614. In the illustrated embodiment, the pedicle screw 612 and the support 614 are the same as the pedicle screw 12 and the support 14 illustrated in FIG. 2 and described above with the exceptions that the opposing arms 638a, 638b of the pedicle screw 612 form a desired structure and the upper portion 628 of the support 614 forms a complementary structure. In the illustrated embodiment, the structure formed by the opposing arms 638a, 638b of the pedicle screw 612 has a circular shape forming an outer grip surface. The complementary structure formed by the upper portion of the support 614 encircles a portion of the outer grip surface. The tool tip 646 has a structure configured to engage the outer grip surface. In operation, the tool tip 646 is inserted over the outer grip surfaces formed by the opposing arms 638a, 638b of the pedicle screw 612 and between the upper portions of the support 614. Advantageously, the tool 644 facilitates removal of the pedicle screw 612 from the support and from the base without the need for human contact, thereby maintaining the sterility of the pedicle screw. As a further advantage of the structure shown in FIG. 11C, the alignment of the tool tip 646 with the outer grip surfaces located between the upper portions of the support 614 facilitates a desired orientation and alignment of the pedicle screw 612 with the tool 644 prior to use of the tool 644 for insertion of the pedicle screw 612 into a surgical area.

While the embodiments shown in FIGS. 11A-11C show specific cooperating structures, it should be appreciated that in other embodiments, other structures can be used including the non-limiting examples of magnetic structures, threaded structures, interlocking structures, keyed structures and the like. It should also be appreciated that the tool can be further configured to engage the inner surfaces of the opposing arms of the pedicle screw, the outer portions of the opposing arms of the pedicle screw and/or internal structures of the pedicle screw. The cooperating structures of the tool and the pedicle screw can also be configured to prevent rotation of the pedicle screw while connected to the tool.

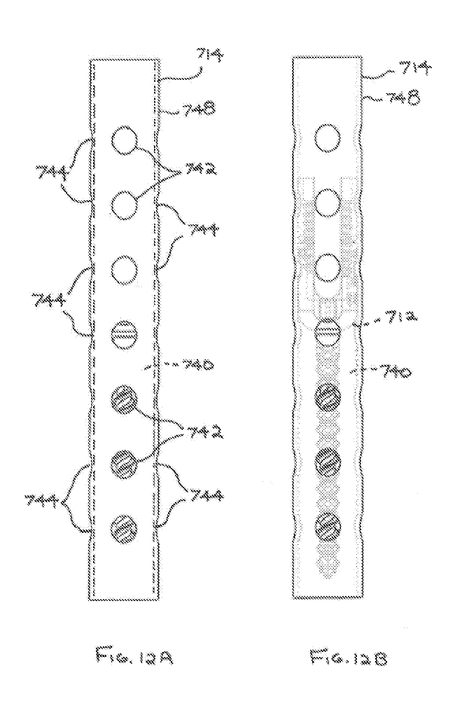

Referring now to FIGS. 12A and 12B, another embodiment of a support 714 is illustrated. The support 714 forms a generally hollow, cylindrically shaped structure and includes an outer circumferential wall 748. The outer circumferential wall 748 defines an interior cavity 740 extending the length of the support 714. A pedicle screw 712 is positioned within the interior cavity 740 of the support 714. The outer circumferential wall 748 includes a plurality of spaced apart apertures 742. The apertures are configured to expose the interior cavity 740. The outer circumferential wall 740 includes a plurality of spaced apart recesses 744 extending horizontally around the support 714. The spaced apart apertures 742 and the spaced apart horizontal recesses 744 are configured for permeability, such as to allow gas sterilization processes, such as for example steam-based or ethylene oxide-based sterilization processes to penetrate the support 714 and access the pedicle screw 712 while the support 714 and the pedicle screw 712 are positioned within the container (not shown for purposes of clarity). With the pedicle screw 712 while the support 714 sterilized while positioned within the container, advantageously the pedicle screw 712 and the support 714 can be sterilized without human contact.

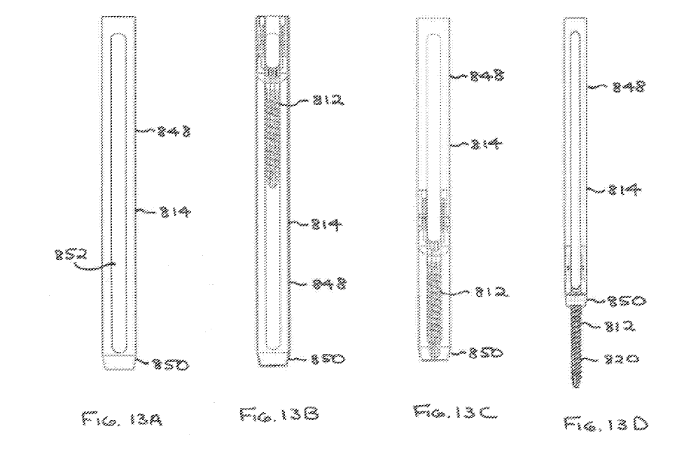

Referring now to FIGS. 13A-13D, another embodiment of a support 814 is illustrated. Referring first to FIG. 13A, the support 814 forms a generally hollow, cylindrically shaped structure and includes an outer circumferential wall 848. The support 814 is configured to house an orthopedic implant device, such as the pedicle screw 812 shown in FIG. 13B. However, the support 814 can also be configured to house other orthopedic implant devices, such as the non-limiting examples of orthopedic screws, bone screws, interbody cages and the like. The support 814 also forms a tapered tip 850. The tapered tip 850 is configured for use in the dissection and manipulation of soft tissue. Optionally the support 814 can include one or more windows 852 configured for the passage of an additional stabilization member, such as a rod.

Referring now to FIG. 13B, the support 814 is illustrated with the pedicle screw 812 in a first position within an interior cavity 849 defined by the outer circumferential wall 848. In certain instances, the container (not shown for purposes of clarity) can be provided with the support 814 and the pedicle screw 812 pre-assembled. However, in other instances, the container and the support 814 with the pedicle screw 812 can be provided separately and subsequently assembled together.

Referring now to FIG. 13C, the support 814 is illustrated with the pedicle screw 812 in a second position near the tip 850 of the support 814. Finally, referring now to FIG. 13D, the support 814 is illustrated with the pedicle screw 812 in a final position. In this position, a threaded shaft portion 820 of the pedicle screw extends from the tip 850 of the support 814 and the support 814 can be used in the dissection and manipulation of soft tissue and installation of the pedicle screw 812. In operation, following placement of the pedicle screw and other implant components, the support 814 can be removed from the pedicle screw 812 via permanent deformation of the support 814 and discarded.

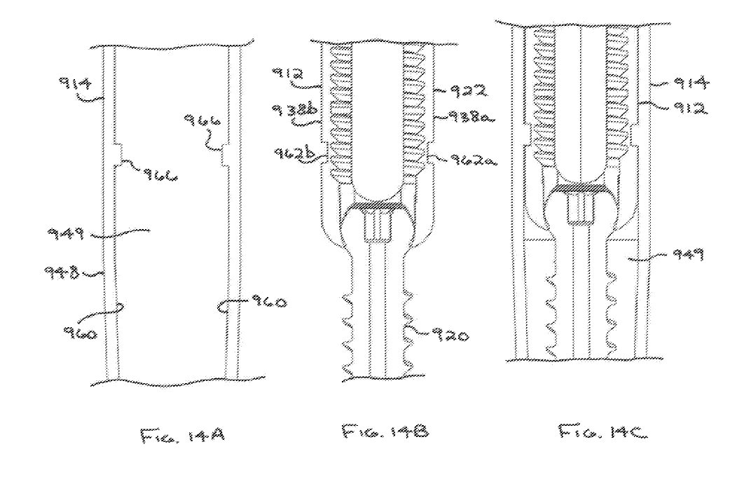

Referring now to FIGS. 14A-14C, another embodiment of a pedicle screw 912 and a support 914 is illustrated. Referring first to FIG. 14A, the support 914 forms a generally hollow, cylindrically shaped structure and includes an outer circumferential wall 948. The outer circumferential wall 948 defines an interior cavity 949 configured to house an orthopedic implant device, such as the pedicle screw 912. However, the support 914 can also be configured to house other orthopedic implant devices, such as the non-limiting examples of orthopedic screws, bone screws, interbody cages and the like. The outer circumferential wall 948 has an interior surface 960. The support 914 includes one or more annular protrusions 966 extending in an inward direction from the interior surface 960 of the outer circumferential wall 948.

Referring now to FIG. 14b, the pedicle screw 912 is illustrated. The pedicle screw 912 includes a threaded shaft portion 920 and a yoke-shaped head portion 922 with opposing arms 938a, 938b. Each of the opposing arms 938a, 938b includes an annular groove 962a, 962b respectively. The annular grooves 962a, 962b have dimensions and a cross-sectional shape configured to approximate the dimensions and cross-sectional shape of the annular protrusion 966.

Referring now to FIG. 14C, the pedicle screw 912 is shown in a secured position within the interior cavity 949 of the support 914. In the illustrated embodiment, the pedicle screw 912 is secured within the interior cavity 949 through an interference fit with the interior surface 960 of the outer circumferential wall 948. However, in other embodiments, other structures, mechanisms and devices can be used to secure the pedicle screw 912 within the interior cavity 949, including the non-limiting examples of mating features, an elastomeric interface sleeve, threaded structures and the like. While the interference fit generally secures the pedicle screw 912 within the support 914, the pedicle screw 912 is further prevented from moving in a longitudinal direction within the support 914 as the annular protrusion 966 is seated in the annular grooves 962a, 962b. It is contemplated that the material forming the support 914 is sufficiently flexible and/or pliable so as to allow a user to pressure the support 914 in a manner to release the annular protrusion 966 from the annular grooves 962a, 962b, thereby allowing the pedicle screw 912 to move in a longitudinal direction.

While the annular grooves 962a, 962b and the annular protrusion 966 have a rectangular cross-sectional shape, it should be appreciated that other shapes can be used sufficient to prevent the pedicle screw 912 from moving in a longitudinal direction within the support 914. It should also be appreciated that in other embodiments, more than one annular groove 962a, 962b and more than one mating annular protrusion 966 can be used. While the embodiment shown in FIGS. 14A-14C illustrates continuous annular grooves 962a, 962b and a continuous annular protrusion 966, in other embodiments, the annular grooves 962a, 962b and the annular protrusion 966 can be formed as discontinuous segments.

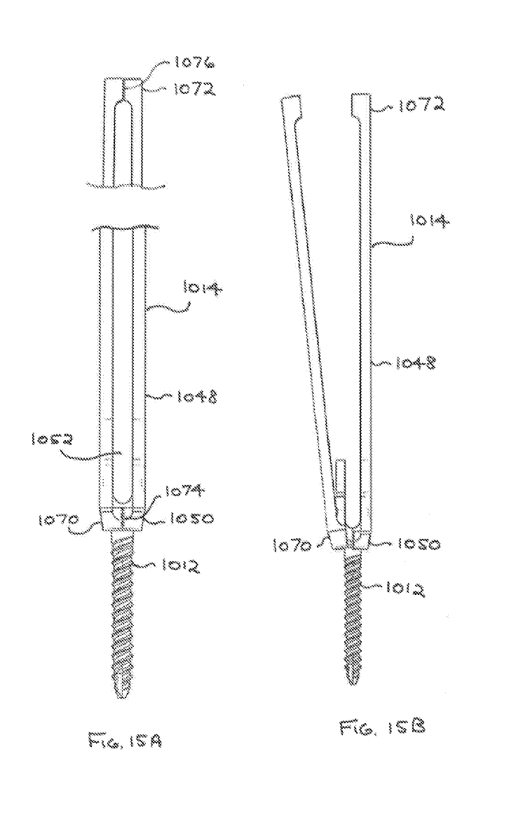

Referring now to FIGS. 15A and 15B, another embodiment of a support 1014 is illustrated. Referring first to FIG. 15A, the support 1014 forms a generally hollow, cylindrically shaped structure and includes an outer circumferential wall 1048. The support 1014 is configured to house an orthopedic implant device, such as the pedicle screw 1012. However, the support 1014 can also be configured to house other orthopedic implant devices, such as the non-limiting examples of orthopedic screws, bone screws, interbody cages and the like. Optionally, the support 1014 can form a tapered tip 1050. The tapered tip 1050 is configured for use in the dissection and manipulation of soft tissue. Optionally, the support 1014 can include one or more windows 1052 configured for the passage of an additional stabilization member, such as a rod.

Referring again to FIG. 15A, the support 1014 has a proximal end 1070 and a distal end 1072. The proximal end 1070 includes a weakened portion 1074 and the distal end 1072 also includes a weakened portion 1076. The weakened portions 1074, 1076 are configured for separation, as shown in FIG. 15B, thereby allowing the pedicle screw 1012 to be separated from the support 1014 once the pedicle screw 1012 is in a desired orientation. In the illustrated embodiment, the weakened portions 1074, 1076 are formed as a line of continuous or discontinuous perforations. In alternate embodiments, the weakened portions 1074, 1076 can be formed from other structures, including the non-limiting example of a small cross-sectional dimension, sufficient to allow the pedicle screw 1012 to be separated from the support 1014 once the pedicle screw 1012 is in a desired orientation.

Referring again to FIG. 15A, the support 1014 is illustrated in a first orientation with the pedicle screw 1012 within the interior cavity 1049 and the weakened portions 1074, 1076 of the proximal end 1070 and a distal end 1072 in a connected arrangement. In certain instances, the container (not shown for purposes of clarity) can be provided with the support 1014 and the pedicle screw 1012 pre-assembled. However, in other instances, the container and the support 1014 with the pedicle screw 1012 can be provided separately and subsequently assembled together.

Referring now to FIG. 15B, the support 1014 is illustrated in a second orientation with the pedicle screw 1012 within the interior cavity 1049 and the weakened portions 1074, 1076 of the proximal end 1070 and the distal end 1072 in a separated arrangement. In this arrangement, a force has been applied to the support 1014 to separate the weakened portions 1074, 1076 such that the weakened portions 1074, 1076 break and the pedicle screw 1012 can be separated from the support 1014. Advantageously, in this manner, the support 1014 may be removed from the pedicle screw 1012 through controlled separation and disassembly and without human contact.

Referring again to FIGS. 15A and 15B, it is contemplated that in other embodiments other structures and manners can be used to accomplish a controlled separation of the orthopedic implant device from the support. The support may be designed to break via forces generated by features on the orthopedic implant device that force the support apart during the final stages of device insertion. As one non-limiting example, if the orthopedic implant device is larger than the tapered tip of the support, the support may separate when the orthopedic implant device is forced through. This may lead to controlled separation and partial or full disassembly of the support from the orthopedic implant device.

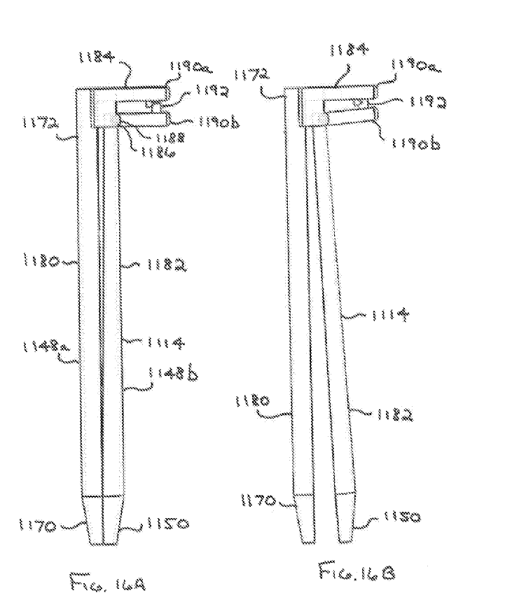

It is also contemplated that controlled separation of the support can be achieved without deformation of the support, but instead by controlled modification of the support. The support can include segmented parts configured for manipulation in a manner which allows the support's contents to exit the support. In one non-limiting example, the segmented parts of the support could articulate in a manner which enlarges an opening, thereby allowing the orthopedic implant device to exit the support. Referring now to FIGS. 16A and 16B, another embodiment of a support 1114 is illustrated. Referring first to FIG. 16A, the support 1114 forms a generally hollow, cylindrically shaped structure and includes opposing portions 1180, 1182. The opposing portions 1180, 1182 each have an outer circumferential wall 1148a, 1148b. The support 1114 is configured to house an orthopedic implant device, such as a pedicle screw (not shown for purposes of clarity). However, the support 1114 can also be configured to house other orthopedic implant devices, such as the non-limiting examples of orthopedic screws, bone screws, interbody cages and the like. Optionally, the support 1114 can form a tapered tip 1150. The tapered tip 1050 is configured for use in the dissection and manipulation of soft tissue. Optionally, the support 1114 can include one or more windows (not shown) configured for the passage of an additional stabilization member, such as a rod.

Referring again to FIG. 16A, the support 1114 has a proximal end 1170 and a distal end 1172. The distal end 1172 includes a framework 1184 configured to facilitate controlled rotation of the proximal end 1170 of the portion 1182 relative to the proximal end 1170 of the portion 1180, thereby allowing a controlled separation of the pedicle screw from the support once the pedicle screw is in a desired orientation. In the embodiment illustrated in FIG. 16A, the framework 1184 includes a hinge 1186 positioned at a pivot point 1188. The hinge 1186 is connected to the portion 1182 of the support 1114 such that the portion 1182 can rotate about the pivot point 1188. Any desired hinge structure can be used sufficient to facilitate controlled pivoting of the portion 1182 about the pivot point 1188. It should also be considered that other structures can be used for controlled pivoting of the portion 1182 about the pivot point 1188 including the non-limiting examples of splay structures, clam-shell structures, bending structures and the like.

Referring again to FIG. 16A, optionally the framework 1184 can include opposing arms 1190a, 1190b configured to actuate controlled rotation of the portion 1182 about the pivot point 1188. However, the support 1114 can include other structures, mechanisms and devices sufficient to actuate controlled rotation of the portion 1182 about the pivot point 1188. The framework 1184 can also include an optional dampening structure 1192. The dampening structure 1192 is configured to provide an opposing spring force to further control the rotation of the portion 1182 about the pivot point 1188. In the illustrated embodiment, the dampening structure 1192 is a compression spring. In the alternate embodiments, the dampening structure 1192 can be other structures, mechanisms and devices sufficient to provide an opposing spring force to further control the rotation of the portion 1182 about the pivot point 1188.

Referring again to FIG. 16A, the support 1114 is illustrated in a first orientation with the opposing portions 1180, 1182 of the support 1114 is a substantially parallel and adjacent arrangement. In certain instances, the container (not shown for purposes of clarity) can be provided with the support 1114 and the pedicle screw pre-assembled. However, in other instances, the container and the support 1114 with the pedicle screw can be provided separately and subsequently assembled together.

Referring now to FIG. 16B, the support 1114 is illustrated in a second orientation with the opposing portions 1180, 1182 of the support 1114 in a separated arrangement. In this arrangement, a force has been applied to the framework 1184 such as to rotate the portion 1182 about the pivot point 1188 and thereby separate the opposing portions 1180, 1182 at the proximal end 1170 of the support 1114 such that the pedicle screw can be separated from the support 1114. Advantageously, in this manner, the support 1114 may be removed from the pedicle screw through controlled separation and disassembly and without human contact.

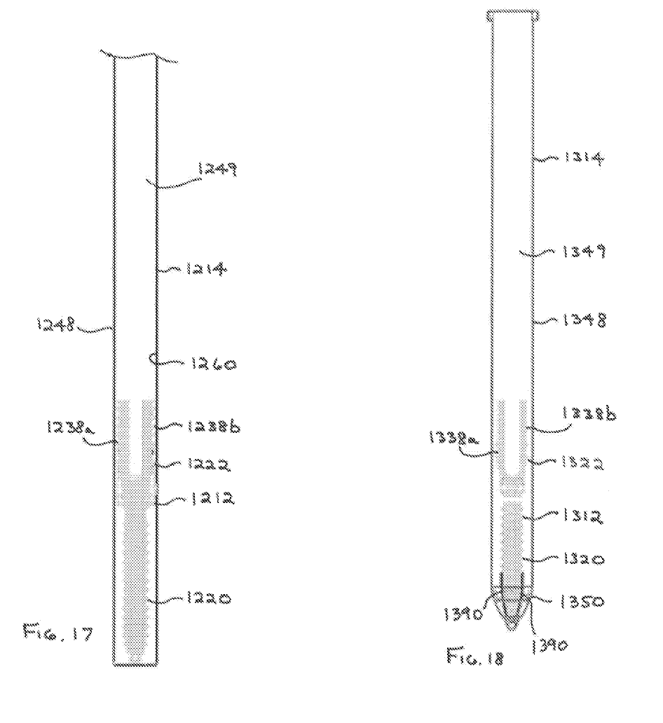

As described above, the pedicle screw or other orthopedic implant device has opposed arms configured for a frictional fit with interior portions of the support. The frictional fit is configured to securely engage the pedicle screw or other orthopedic implant device. However, in other embodiments, instead of a controlled separation of the pedicle screw or other orthopedic implant device from the support, it is contemplated the orthopedic implant device may be delivered through the support freely or with slight interference. Referring now to FIG. 17, another embodiment of a support is shown generally at 1214. The support 1214 forms a generally hollow, cylindrically shaped structure and includes an outer circumferential wall 1248. The outer circumferential wall 1248 defines an interior cavity 1249 configured to house an orthopedic implant device, such as the pedicle screw 1212. However, the support 1214 can also be configured to house other orthopedic implant devices, such as the non-limiting examples of orthopedic screws, bone screws, interbody cages and the like. The outer circumferential wall 1248 has an interior surface 1260.

Referring again to FIG. 17, the pedicle screw 1212 is illustrated. The pedicle screw 1212 includes a threaded shaft portion 1220 and a yoke-shaped head portion 1222 with opposing arms 1238a, 1238b. The pedicle screw 1212 is shown within the interior cavity 1249 of the support 1214. In the illustrated embodiment, the pedicle screw 1212 is not secured within the interior cavity 1249 through an interference fit with the interior surface 1260 of the outer circumferential wall 1248. Rather, the pedicle screw 121 is configured for delivery through the interior cavity 1249 of the support 1214 with little or no interference. The unencumbered delivery of the pedicle screw 1212 through the interior cavity 1249 of the support 1214 advantageously facilitates delivery of the pedicle screw 121 through a large section of soft tissue, as is frequently performed during minimally invasive surgery, without the need for human contact, thereby maintaining the sterility of the pedicle screw.

Referring again to the embodiment illustrated in FIG. 17, the interior surface 1260 of the outer circumferential wall 1248 has a smooth surface configured to facilitate an unencumbered delivery of the pedicle screw 1212 through the interior cavity 1249 of the support 1214. In other embodiments, the interior surface 1260 of the outer circumferential wall 1248 can have other configurations, such as for example, a surface coating configured to facilitate an unencumbered delivery of the pedicle screw 1212 through the interior cavity 1249 of the support 1214. In still other configurations, the outer circumferential wall 1248 can have other configurations, such as for example, discontinuous interior surfaces of the outer circumferential wall 1248 to facilitate an unencumbered delivery of the pedicle screw 1212 through the interior cavity 1249 of the support 1214.

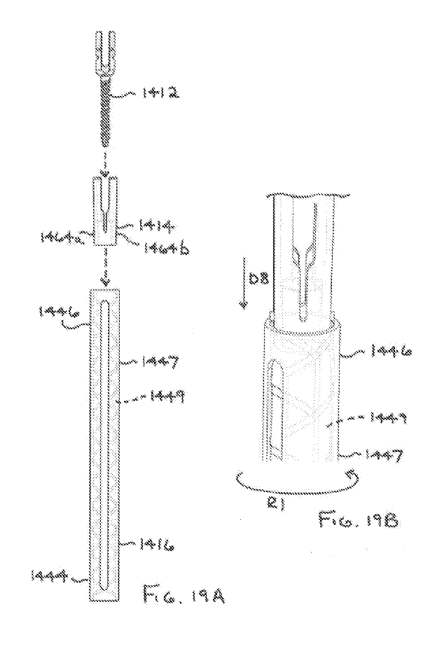

In certain instances, the orthopedic implant device contained within the support can be larger than a tapered end of the support. In these instances, the support can be permanently and/or elastically deformed to allow the exit of the orthopedic implant device. To help facilitate the deformation of the support and the exit of the orthopedic implant device, there may be features incorporated into the support. Referring now to FIG. 18, another embodiment of a support is shown generally at 1314. The support 1314 forms a generally hollow, cylindrically shaped structure and includes an outer circumferential wall 1348. The outer circumferential wall 1348 defines an interior cavity 1349 configured to house an orthopedic implant device, such as the pedicle screw 1312. However, the support 1314 can also be configured to house other orthopedic implant devices, such as the non-limiting examples of orthopedic screws, bone screws, interbody cages and the like. The support 1314 forms a tapered tip 1350.

Referring again to FIG. 18, the pedicle screw 1312 is illustrated. The pedicle screw 1312 includes a threaded shaft portion 1320 and a yoke-shaped head portion 1322 with opposing arms 1338a, 1338b. In the illustrated embodiment, the opposing arms 1338a, 1338b form a diameter that is larger than a diameter of the outer circumferential wall 1348 of the support 1314 and also larger than a diameter of the tapered tip 1350. Accordingly, the support 1314 is equipped with a plurality of features 1390 configured to help facilitate the deformation of the support 1314 and the exit of the orthopedic implant device 1312.

Referring again to the embodiment illustrated in FIG. 18, the features 1390 include slits extending longitudinally along the taper tip 1350. The slits 1390 are configured to allow the tapered tip 1350 to expand as the orthopedic implant device 1312 exits the support 1314. Any desired number of slits 1390 can be used sufficient to allow the tapered tip 1350 to expand as the orthopedic implant device 1312 exits the support 1314. While the slits 1390 are shown in the tapered tip 1350, in other embodiments, the slits 1390 can extend along the support in a longitudinal direction a distance sufficient to allow the tapered tip 1350 to expand as the orthopedic implant device 1312 exits the support 1314. While the features 1390 are described above a slits, alternatively, the features can be any desired structure, including the non-limiting examples of holes, perforations, cuts and the like, sufficient to allow the tapered tip 1350 to expand as the orthopedic implant device 1312 exits the support 1314.

Referring again to FIG. 18, the features 1390 advantageously allow the tapered tip 1350 to expand as the orthopedic implant device 1312 exits the support 1314, without the need for human contact, thereby maintaining the sterility of the pedicle screw.

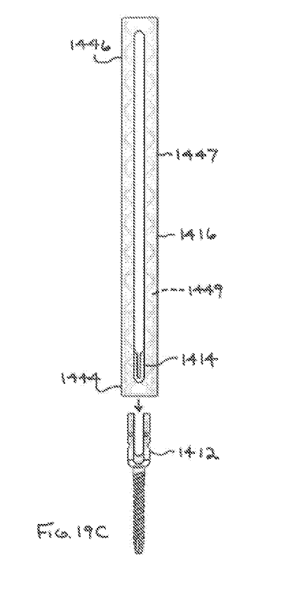

Referring now to FIGS. 19A-19C, another embodiment of a portion of a container is shown generally at 1460. The container 1460 includes a base 1416, a carrier 1414 and an orthopedic implant device 1412. The base 1416 is generally hollow and cylindrical in shape. The base 1416 includes a lower portion 1444, an upper portion 1446 and an outer circumferential wall 1447. Referring again to FIGS. 19A-19C, the outer circumferential wall 1447 defines an interior cavity 1449 extending the length of the base 1416. The base 1416 also includes a plurality of helical grooves 1462a, 1462b provided on an inner surface of the outer circumferential wall 1447. In the illustrated embodiment, the helical grooves 1462a, 1462b are the same as, or similar to, the helical grooves 62a, 62b described above and shown in FIGS. 4 and 5. Alternatively, the helical grooves 1462a, 1462b can be different than the helical grooves 62a, 62b.

Referring again to the embodiment shown in FIGS. 19A-19C, the carrier 1414 is the same as, or similar to the upper portion 28 of the support 14 described above and shown in FIG. 2 with the exception that the carrier 1414 includes a plurality of tabs 1464a, 1464b configured to extend into the helical grooves 1462a, 1462b of the base 1416. As a result of rotation of the base 1416, as represented by direction arrow R1, the tabs 1464a, 1464b of the carrier 1414 cooperate with the helical grooves 1462a, 1462b in the base 1416 to effect a controlled longitudinal movement of the carrier 1414, as represented by direction arrow D8.

Referring again to FIGS. 19A-19C, the orthopedic implant device 1412 is the same as, or similar to, the pedicle screw 12 described above and shown in FIG. 2. In other embodiments, the orthopedic implant device 1412 can be different from the pedicle screw 12. The orthopedic implant device 1412 is arranged within the interior cavity 1449 of the base 1416, such that the controlled longitudinal movement of the carrier 1414 results in corresponding controlled longitudinal movement of the orthopedic implant device 1412.

Referring now to FIG. 19C, once the carrier 1414 and the orthopedic implant device 1412 reach the bottom of the base 1416, the carrier 1414 is retained within the interior cavity 1449 of the base 1416, but the orthopedic implant device 1412 is permitted to pass through the carrier 1414 and is inserted into the desired anatomy through controlled movement. Advantageously, the orthopedic implant device 1412 is guided through the base 1416 and the orthopedic implant device 1312 exits the carrier 1414 and the base 1416 without the need for human contact, thereby maintaining the sterility of the orthopedic implant device 1412.

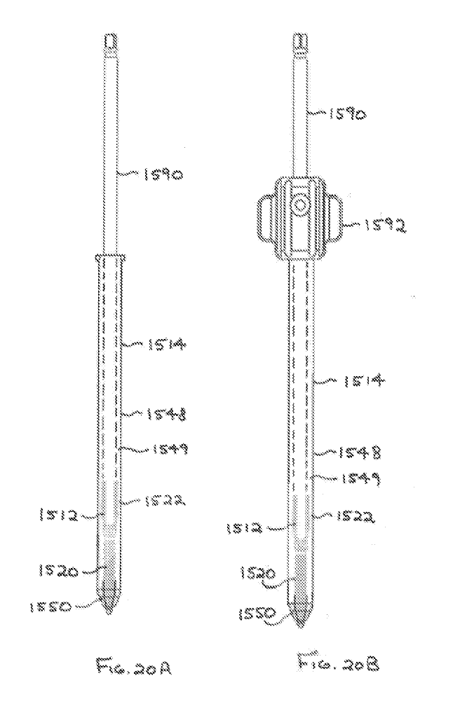

Referring now to FIGS. 20A and 20B, another embodiment of a support is shown generally at 1514. The support 1514 forms a generally hollow, cylindrically shaped structure and includes an outer circumferential wall 1548. The outer circumferential wall 1548 defines an interior cavity 1549 configured to house an orthopedic implant device, such as the pedicle screw 1512. However, the support 1514 can also be configured to house other orthopedic implant devices, such as the non-limiting examples of orthopedic screws, bone screws, interbody cages and the like. The support 1514 forms a tapered tip 1550.

Referring again to FIGS. 20A and 20B, the pedicle screw 1512 is illustrated. The pedicle screw 1512 includes a threaded shaft portion 1520 and a yoke-shaped head portion 1522. The taper tip 1550 includes a plurality of features 1590 in the event portions of the pedicle screw 1515 are larger than the tapered tip 1550, as discussed above.

Referring again to FIGS. 20A and 20B, a tool 1590 is configured to extend into the interior cavity 1549 of the support 1514 and is also configured for connection with the pedicle screw 1512. The tool 1590 is further configured for use in the implantation of the pedicle screw 1512 in the surgical site, that is, the tool 1590 is configured to move the pedicle screw 1512 through the support 1514 and into the surgical site. After insertion of the pedicle screw 1512 into the surgical site, the support 1514 and the tool 1590 are removed from the surgical site. The tool 1590 can have different forms, including the non-limiting examples of screw drivers, inserters, handles, tamps, levers and the like.

Referring now to FIG. 20B, it is within the contemplation of the invention that the support 1514 and/or tool 1590 can be equipped with one or more optional instruments 1592 configured to provide information to the user. Non-limiting examples of instruments 1592 can include depth gages, relative depth markings, k-wire components and the like. After insertion of the pedicle screw 1512 into the surgical site, the support 1514, tool 1590 and instruments 1592 are removed from the surgical site.

The support 1514 and the tool 1590 advantageously facilitate insertion of the pedicle screw 1512, without the need for human contact, thereby maintaining the sterility of the pedicle screw.

The principle and mode of operation of the combined protective container and delivery device for a pedicle screw have been explained and illustrated in its preferred embodiments. However, it must be understood that the combined protective container and delivery device for a pedicle screw may be practiced otherwise than as specifically explained and illustrated without departing from its spirit or scope.

What is claimed is:

1. A combined protective container and delivery device for an orthopedic implant device comprising:
an orthopedic implant device configured for insertion into a surgical site;
a support configured to receive the orthopedic implant device;
a base configured to receive the orthopedic implant device and the support; and
a cover configured to form a sealed container to prevent the ingress of contaminants to the orthopedic implant device and the support;
wherein the sealed container is configured for removal and insertion of the orthopedic implant device without manual handling of the orthopedic implant device.

2. The combined protective container and delivery device of claim 1, wherein the orthopedic implant device is configured for a friction fit with the support.

3. The combined protective container and delivery device of claim 1, wherein the base includes a plurality of helical grooves.

4. The combined protective container and delivery device of claim 3, wherein the plurality of helical grooves are configured to receive tabs extending from an internal platform.

5. The combined protective container and delivery device of claim 4, wherein the internal platform is configured for longitudinal movement as a result of rotation of a portion of the base.

6. The combined protective container and delivery device of claim 1, wherein a spring element is positioned within the base and configured to urge an internal platform in contact with the support.

7. The combined protective container and delivery device of claim 6, wherein the support is retained within the base by a cap.

8. The combined protective container and delivery device of claim 1, wherein the base includes a linear translation device configured to move the support in a longitudinal direction.

9. The combined protective container and delivery device of claim 1, wherein the base includes one or more internal paths configured to receive detents extending from the support.

10. The combined protective container and delivery device of claim 1, wherein the orthopedic implant device includes a recess configured to receive a mating tool.

11. The combined protective container and delivery device of claim 1, wherein the support includes a plurality of apertures configured to expose and interior cavity, wherein the apertures are configured to facilitate gas sterilization processes.

12. The combined protective container and delivery device of claim 1, wherein the support includes at least one window configured for the passage of an additional stabilization member.

13. The combined protective container and delivery device of claim 1, wherein the support includes at least one annular protrusion and the orthopedic implant device includes at least one mating annular groove.

14. The combined protective container and delivery device of claim 1, wherein the support includes opposing portions connected together by break-away features, the break-away features configured for removal of the opposing portions.

15. The combined protective container and delivery device of claim 1, wherein the support includes opposing portions, and at least one of the opposing portions is configured to rotational movement relative to each other thereby facilitating movement of the orthopedic implant device from the support.

16. The combined protective container and delivery device of claim 1, wherein the support includes a tip having a plurality of features configured to allow the tip to expand as the orthopedic implant device moves through the tip.

17. The combined protective container and delivery device of claim 16, wherein the tip is tapered.

18. The combined protective container and delivery device of claim 1, wherein the orthopedic implant device is configured for movement through the support to the surgical site and the support is configured to remain in the base.

19. The combined protective container and delivery device of claim 1, wherein a tool is configured to extend into the support and further configured to move the orthopedic implant device through the support to the surgical site.

20. The combined protective container and delivery device of claim 1, wherein the support includes an instrument configured to provide information to the user.
